(19) United States
(12) Patent Application Publication
(10) Pub. No.: US 2021/0193269 A1
(43) Pub. Date: Jun. 24, 2021

(54) Method for Assessing Classification Annotations Assigned to DNA Sequences of Organisms

(71) Applicant: SmartGene GmbH, Zug (CH)

(72) Inventors: Stefan Emler, Zürich (CH); Pierre-André Michel, Genève (CH)

(21) Appl. No.: 17/188,546

(22) Filed: Mar. 1, 2021

Related U.S. Application Data
(63) Continuation of application No. 15/616,873, filed on Jun. 7, 2017.
Application No. 15/616,873 is a continuation of application No. 12/744,573, filed on May 25, 2010, now abandoned.

(51) Int. Cl. G16B 50/00

(52) U.S. Cl. CPC G16B 50/00; G16B 30/00

(57) ABSTRACT

The present invention enables accurate identification of organisms by analyzing their DNA sequences and, based on their DNA sequences, assessing classification annotations, such as taxonomic, systematic, or functional annotations. Sequence-based identification of life forms as described herein can be used for diagnostic purposes, for example. Further, the techniques disclosed herein offer advantages over conventional culture-based techniques. Example embodiments are related to methods for assessing classification annotations assigned to DNA sequences of organisms. One example embodiment includes a method of identifying a centroid DNA sequence of one or more organisms. The method includes obtaining a plurality of DNA sequences from one or more organisms, annotating each DNA sequence with a classification annotation, and grouping the plurality of DNA sequences into a plurality of groups. Further, the method includes selecting a group of the plurality of groups and determining, for the selected group, a centroid sequence.

### CROSS-REFERENCE TO RELATED APPLICATIONS

The present application is a continuation of U.S. patent application Ser. No. 15/616,873 filed Jun. 7, 2017, which is a continuation of U.S. patent application Ser. No. 12/744,573 filed May 25, 2010, which is a national stage entry of PCT/CH2007/000599 filed Nov. 29, 2007, the contents of each of which are hereby incorporated by reference.

### SEQUENCE LISTING STATEMENT

A computer readable form of the Sequence Listing is filed with this application by electronic submission and is incorporated into this application by reference in its entirety. The Sequence Listing is contained in the file created on Oct. 30, 2017, having the file name “14-358-US-CON2_SequenceListing_ST25.txt” and is 5 kB in size.

### FIELD OF THE INVENTION

The present invention relates to a computer-implemented method and a computer system for assessing classification annotations assigned to DNA sequences. Specifically, the present invention relates to a computer-implemented method and a computer system for assessing classification annotations assigned to DNA sequences stored in a database.

### BACKGROUND OF THE INVENTION

Sequence-based identification of life forms is increasingly used for diagnostic purposes. Being independent of growth and metabolism, this method offers significant advantages over conventional culture-based techniques in terms of speed and accuracy. Conserved genes present in all bacteria or fungi are amplified and subsequently sequenced using automated sequencing techniques. The sequences obtained are then compared to references in a database. Thus, even rare, unexpected or unusual isolates can be rapidly identified and classified. Sequence analysis can be applied to all conserved genes of all life-forms, particularly to microorganisms such as bacteria and fungi. Sequence-based identification of microorganisms relies on comparison of the sample signature sequence to a database containing reference sequences representing all relevant genus and species. It is therefore important that a reference database fulfills the following requirements:
1) Accurate sequence: the database contains correct sequences of the requested target, no sequencing errors, no reading flaws, no artificial gaps, insertions, no vector sequences.
2) Correct classification annotation (i.e. naming of entries): sequences are correctly annotated (e.g. species names) and this information is updated with regard to changes in taxonomy.
3) Representative: the database represents all relevant life-forms, e.g. genus and species, including their genetic variants (intra-species, intra-genomic).
4) Up-to-date: the references are up-to-date with regard to recently described species and potential changes in taxonomy (see also 2).

Currently there is no single reference database which fulfils all these requirements. However, because the quality of results of sequence comparisons greatly depends on the available references, it is crucial that these databases be as reliable as possible. In general, scientists add entries to public repositories which are of fair quality in terms of sequence content and annotation (e.g. species name). Nevertheless, there are many sequencing errors or incorrect annotations with regard to current taxonomy. Annotation errors occur, for example, when sequences are submitted along with incorrect information about the organism or gene from which the sequence has been derived, or with species names which are not up-to-date (e.g. when species have been reclassified taxonomically, as is often the case for bacteria). When a sample sequence is searched against a reference database, the resulting list usually displays indistinguishably correct and incorrect matches, leaving it up to the expertise of the user to determine references which were identified correctly or incorrectly. Thus, a correct sequence with an incorrect annotation could appear on top of the list of matches and, therefore, indicate an erroneous identification of a bacterium, for example. Because sequence-based pathogen identification is becoming nowadays part of the routine work in medical diagnostic, veterinary and industry laboratories, there is a need to render sequence database searches and comparisons easy and reliable, e.g. for identifying a bacterial or fungal species or a virus subtype, or for matching any unknown organism to a database of well characterized organisms. Particularly, the results of searching and comparing sequence similarity need to be provided adequately with regard to the expertise of routine lab technicians, who in general do not have a research background or extensive training in bio-informatics or (micro-) organism taxonomy.

US 2007/0083334 describes systems and methods for annotating biomolecular sequences. Subsequent to sequence alignment(s), biomolecular sequences are computationally clustered according to a progressive homology range using one or more clustering algorithms. A biomolecular sequence is considered to belong to a cluster, if the sequence shares an alignment-based sequence homology above a certain threshold to one member of the cluster. According to US 2007/0083334, computational clustering can be effected using any commercially available alignment software including a local homology algorithm. For example, a group exhibits a certain degree of homology, if the nucleic acids are 90% identical to one another.

US 2007/0134692 describes an alignment-based method and system for updating probe array annotation data. One or more clusters are generated by transcript across datasets retrieved from one or more sources. One or more probe sequence is aligned to a representative sequence from one or more of the clusters. The representative sequence is aligned to a genome sequence and the genome sequence is annotated with probe location information. The aligned probe sequences are mapped to the genome sequence using the alignment of the representative sequence and genome sequence. A score is computed using a number associated with the aligned probe sequences and a number associated with the probe location formation associated with a region of the genome sequence that corresponds to the aligned representative sequence. Redundant entries may be eliminated using the clustering method. For example, if the alignment of transcripts in a cluster overlap by >97% over their entire length, then they are determined to be redundant and only the longest sequence is kept in the cluster.

### SUMMARY OF THE INVENTION

It is an object of this invention to provide a computer-implemented method and a computer system for assessing (and re-assessing) classification annotations, including taxonomic, systematic and/or functional annotations, assigned to DNA sequences. In particular, it is an object of the present invention to provide a computer-implemented method and a computer system for assessing qualitatively the classification annotations such that erroneous and/or doubtful annotations become easily apparent to lab technicians who do not have extensive experience or training in bio-informatics or (micro-) organism taxonomy.

According to the present invention, these objects are achieved particularly through the features of the independent claims. In addition, further advantageous embodiments follow from the dependent claims and the description.

According to the present invention, the above-mentioned objects are particularly achieved in that, for assessing classification annotations (including taxonomic, systematic and/or functional annotations) assigned to DNA sequences stored in a database, e.g. a reference database, the DNA sequences are grouped by species using established classification schemes for taxonomic, systematic and/or functional classification. Subsequently, for pairs of the DNA sequences, determined is in each case a measure of distance between the respective DNA sequences. The measure of distance is determined by aligning automatically the respective DNA sequences and defining the measure of distance based on a score of similarity between the aligned DNA sequences. For example, the measure of distance between two DNA sequences is calculated as a complementary value to the score of similarity, e.g. by subtracting a weighted score of similarity from one. For example, the weighted score of similarity is calculated by dividing the score of similarity between the two DNA sequences through the smaller length of the two DNA sequences. Subsequently, determined is a centroid sequence having the shortest aggregate measure of distance to the DNA sequences. Preferably, within a defined group of DNA sequences, e.g. DNA sequences related to one species, the centroid sequence is the one of these DNA sequences that has the shortest accumulated measure of distance to the other DNA sequences in the group. Alternatively, the centroid sequence is an entirely virtual object, calculated to have the lowest average measure of distance to all the DNA sequences to be considered. It should be noted that within the present context, the term “centroid sequence” is used to include a centroid object representative of an actual DNA sequence as well as a centroid object representative of a virtual object. Assigned to each one of the DNA sequences to be considered is the measure of distance between the respective one of the DNA sequences and the centroid sequence, as a quantitative confidence level for the classification annotation of the respective one of the DNA sequences. Preferably, the confidence levels are stored in the database assigned to the respective annotation and DNA sequence which match a known species or genus name. The assessment and rating of the classification annotations with these confidence levels makes it possible to provide to a user an indication of the degree of representativeness of a DNA sequence for a particular species. For example, when a user performs a query on the database, with each entry in the list of matching reference sequences a field is displayed for the user, indicating the level of confidence that the respective DNA sequence is representative for that particular species and/or genus. Depending on the embodiment, the quantitative confidence level, i.e. the measure of distance to a centroid sequence, is a numeric value or a qualitatively descriptive value derived from the numeric value. For numeric confidence levels, a small measure of distance indicates a trustworthy annotation, whereas with a greater distance, the entry should be considered more carefully with regards to providing a valid identification.

In a preferred embodiment, the measure of distance is determined between DNA sequences within a species and centroid sequences are determined for the DNA sequences within each of the species. Furthermore, outliers are defined within the species, whereby the outliers are those DNA sequences that have the greatest measures of distance to the centroid sequence of the respective species. For example, one or more outliers are defined based on a maximum distance threshold, a defined deviation from an average measure of distance, or a defined number or quantity of DNA sequences having the largest measure of distance from the centroid sequence. For outliers which have a smaller measure of distance to a centroid sequence of another species, the annotations are marked as incorrect, e.g. by setting a respective indicator in the database.

In an embodiment, an edge-weighted graph is generated from the scores of similarity between the DNA sequences. In this graph, the DNA sequences are nodes in the graph, and the nodes are connected, if the score of similarity between the respective DNA sequences is positive (unalignable and dissimilar sequences are assigned a similarity of zero). The measure of distance between the respective DNA sequences is assigned in each case an edge weight. For the nodes in the graph, local connectivity densities (number of connections to other nodes) are computed. Clusters of nodes are defined through progressive aggregation to local connectivity density maxima, whereby the measure of distance between DNA sequences associated with nodes within a cluster (intra-cluster distance) is significantly shorter than an average measure of distance between the DNA sequences associated with the nodes of the graph (average graph distance).

In a further embodiment, a cluster threshold is received in the computer from the user, e.g. in response to the user viewing the graph shown on a display. Subsequently, the clusters of nodes are defined by applying the cluster threshold as a maximum intra-cluster distance. Thus, nodes associated with DNA sequences having a measure of distance greater than the maximum intra-cluster distance are not included in the cluster. After application of the cluster threshold, the graph is shown on the display. By selecting different cluster thresholds, the user is enabled to select a level of granularity of the graph in the sense that with a relatively high value of the cluster threshold, the graph is typically a coherent structure connecting all nodes, whereas for smaller cluster thresholds, the graph typically disintegrates into multiple clusters.

Preferably, in the graph-based approach, the DNA sequence associated with the node having the highest connectivity density in a cluster, i.e. the highest number of connections to other nodes, is defined the centroid sequence of that cluster.

In an embodiment, the classification annotation associated with a centroid sequence is assigned to DNA sequences associated with that centroid sequence. Specifically, the annotation of the centroid of a particular cluster is assigned to DNA sequences associated with the nodes of that cluster. Preferably, this annotation does not overwrite the existing classification annotation of a DNA sequence but is added as a recommendation which can be displayed to users.

In addition to a computer-implemented method and a computer system for assessing classification annotations assigned to DNA sequences stored in a database, the present invention also relates to a computer program product including computer program code means for controlling one or more processors of a computer, such that the computer performs the method, particularly, a computer program product including a computer readable medium containing therein the computer program code means.

### BRIEF DESCRIPTION OF THE DRAWINGS

The present invention will be explained in more detail, by way of example, with reference to the drawings in which:

FIG. 1 shows a block diagram illustrating schematically an exemplary configuration of a computer-based system for practicing embodiments of the present invention, said configuration comprising a computer system with a database, and said configuration being connected to a data entry terminal via a telecommunications network.

FIG. 2 shows a flow diagram illustrating an exemplary sequence of steps for rating classification annotations assigned to DNA sequences.

FIG. 3 shows a flow diagram illustrating an exemplary sequence of steps for determining one or more centroid sequences.

FIG. 4 shows an example of a cluster of DNA sequences related to a centroid sequence.

FIG. 5 shows an alignment of 11 exemplary variations of DNA sequences related to a species.

FIG. 6 shows an example of a user interface showing to a user possible matches for a sample sequence, each possible match being indicated with a confidence level (dist).

### DETAILED DESCRIPTION OF THE PREFERRED EMBODIMENTS

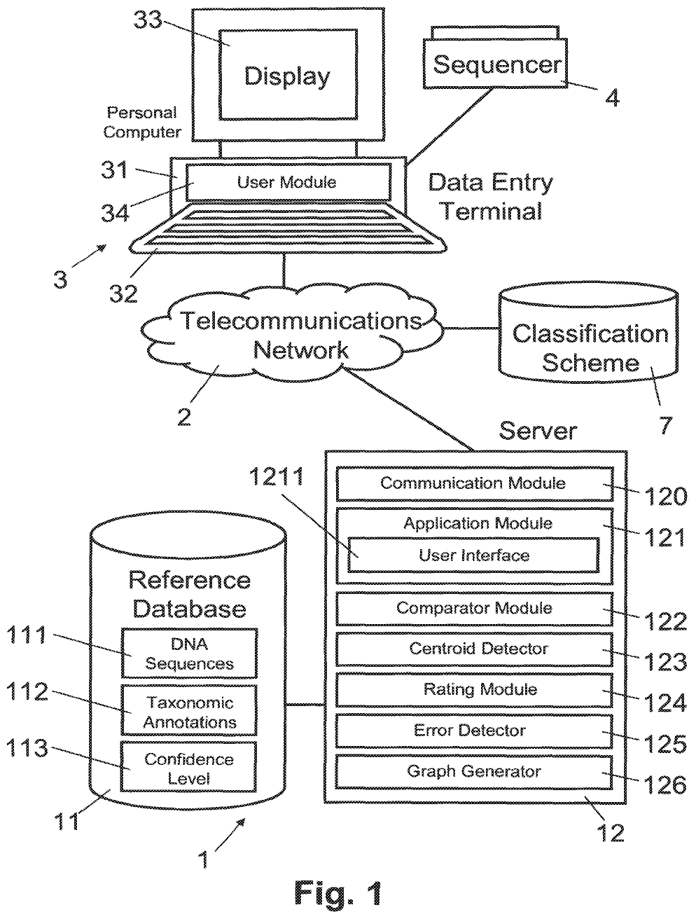

In FIG. 1, reference numeral 3 refers to a data entry terminal. As illustrated in FIG. 1, the data entry terminal 1 includes a personal computer 31 with a keyboard 32 and a display monitor 33, for example.

As is illustrated in FIG. 1, the data entry terminal 3 is connected to computer system 1 through telecommunications network 2. Preferably, the telecommunications network 2 includes the Internet and/or an Intranet, making computer system 1 accessible as a web server through the World Wide Web or within a separate IP-network, respectively. Telecommunications network 2 may also include another fixed network, such as a local area network (LAN) or an integrated services digital network (ISDN), and/or a wireless network, such as a mobile radio network (e.g. Global System for Mobile communication (GSM) or Universal Mobile Telephone System (UMTS)), or a wireless local area network (WLAN). In a variant, at least one data entry terminal 3 is connected directly to computer system 1.

Computer system 1 includes one or more computers, each having one or more processors. Moreover, the computer system 1 comprises a (reference) database 11 including stored entries of reference DNA sequences 111. As illustrated schematically in FIG. 1, computer system 1 includes different functional modules, namely a communication module 120, an application module 121, a comparator module 122, a centroid detector 123, a rating module 124, an error detector 125, and a graph generator 126. Database 11 is implemented on a computer shared with the functional modules or on a separate computer. As is illustrated schematically in FIG. 1, reference database 11 includes classification annotations 112, including taxonomic, systematic and/or functional annotations, associated with DNA sequences 111. Typically, the content of reference database 11 includes entries related to DNA sequences retrieved and obtained from different (public or private) DNA sequence databases. The communication module 120 includes conventional hardware and software elements configured for exchanging data via telecommunications network 2 with one or more data entry terminals 3. The application module 121 is a programmed software module configured to provide users of the data entry terminal 3 with a user interface 1211. Preferably, user interface 1211 is provided through a conventional Internet browser such as Microsoft Explorer or Mozilla Firefox. The comparator module 122, the centroid detector 123, the rating module 124, the error detector 125, and the graph generator 126 are preferably programmed software modules executing on a processor of computer system 1.

Reference numeral 7 refers to a (networked) classification scheme database accessible to computer system 1 via telecommunications network 2. The classification scheme database includes current established classification schemes for the taxonomic, systematic and/or functional classification of DNA sequences of life forms. The classification schemes are non-static and subject to change and/or addition.

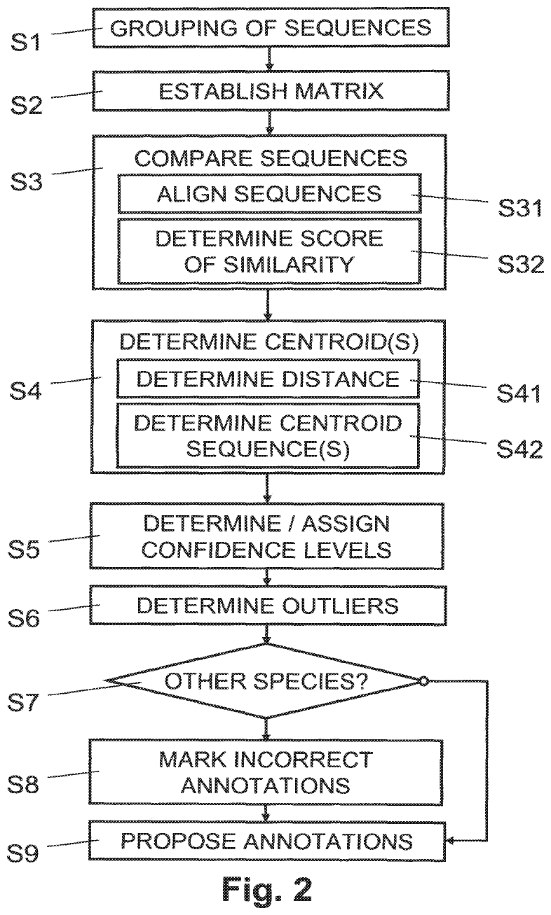

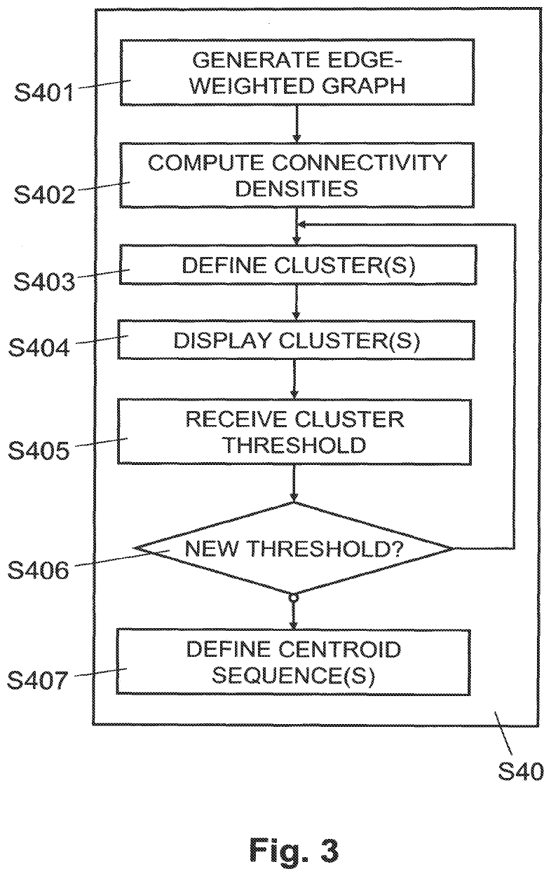

In the following paragraphs the functionality of the functional modules is described with reference to FIGS. 2 and 3.

In step S1, based on their respective classification annotations 112, the comparator module 122 groups by species the DNA sequences 111 stored in reference database 11 using current established classification schemes available from the classification scheme database 7. The grouping of the DNA sequences is performed for all the DNA sequences 111 or for a selected group of the DNA sequences 111. For example, the comparator module 122 is activated by an operator command a user request. In an embodiment, the comparator module 122 is activated periodically or automatically whenever a change, addition or update occurred to the classification scheme 7, or a defined number of new DNA sequences 111 have been entered (added) in the reference database 11 and/or associated with a species. Consequently, the classification annotations 112 assigned to DNA sequences 111 are assessed and re-assessed continuously and repeatedly, e.g. depending on changes in the reference database 11 and/or the classification scheme database 7.

In step S2, the comparator module 122 generates a matrix for comparing the (selected) DNA sequences 111. Depending on the embodiments, one common matrix is generated for all the DNA sequences 111, or different matrices are generated for each species.

In step S3, the comparator module 122 compares the (selected) DNA sequences 111. First the respective DNA sequences are aligned automatically in step S31.

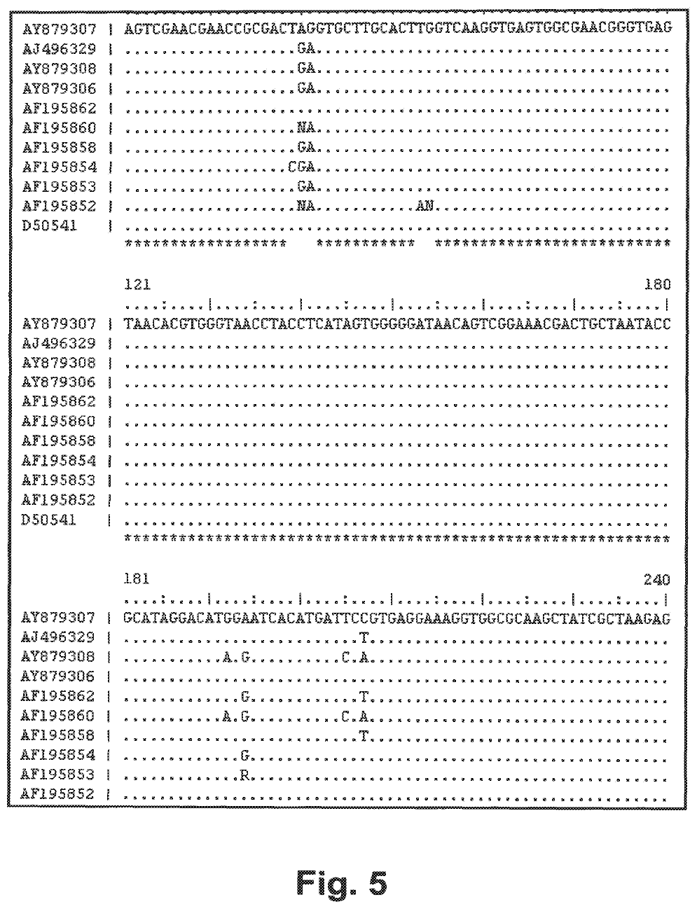

FIG. 5 shows an example of an alignment of eleven sequences (e.g. bacterial ribosomal sequences, commonly used for bacterial sequence-based species identification and taxonomy) representing “Abiotrophia defectiva”. As can be seen in FIG. 5, these sequences are not identical; they carry differences or mutations which may either reflect sequencing errors or reflect true intraspecies or intragenomic variations. From the alignment of these sequences, it becomes apparent that these variations are often grouped and that it is possible to determine a sequence which represents best the alignment (here AY879307) and, therefore, also the bacterial species with the annotation “Abiotrophia defectiva”, with regard to all published “Abiotrophia defectiva” 16S rDNA sequences that are considered.

In step S32, the comparator module 122 determines a score of similarity between the aligned DNA sequences 111, e.g. a score expressed as a percentage of sequence correspondence. The scores of similarity between the (selected) DNA sequences 111 are stored in the matrix. It must be emphasized that the score of similarity may be determined using various different alignment algorithms, e.g. pair wise, global, local, weighted and/or profile-based alignment algorithms, and taking into consideration other elements from the annotations than the classification information.

In step S4, centroid sequence(s) C are determined for the (selected) DNA sequences 111. First, in step S41, the comparator module 122 determines a measure of distance between the respective (selected) DNA sequences 111. The measure of distance is determined based on the scores of similarity between the aligned DNA sequences 111. In an embodiment, the measure of distance is determined between DNA sequences 111 within a species. Preferably, the measures of distance between the (selected) DNA sequences 111 are stored in the matrix.

For example, the measure of distance dist(x, y) between two DNA sequences x and y is calculated by determining a complementary value of the score of similarity, e.g. dist(x,y)=1−score(x,y). Preferably, the measure of distance dist(x, y) between two DNA sequences x and y is calculated by determining a complementary value of a weighted score of similarity e.g. by subtracting the weighted score of similarity from one, the weighted score of similarity being calculated by dividing the score of similarity between the two aligned DNA sequences x, y through the smaller length lₓ, l_{y} of the two DNA sequences x, y:

$$dist ( x , y ) = 1 - \frac{score ( x , y )}{min ( {l}_{x} , {l}_{y} )} .$$

In step S42, based on the measures of distance, the centroid detector 123 determines the centroid sequence(s) C for the (selected) DNA sequences 111. Essentially, for each of the grouped species, the centroid sequence C is the DNA sequence in the group which has the shortest aggregate measure of distance D to the other DNA sequences in the group. Alternatively, a centroid sequence C is defined as a virtual object which is determined to have the shortest possible measure of distance to all the DNA sequences in the group. In other words, c is the centroid sequence of a set of sequences S, if for all N sequences s in set S different from c:

D(c)<D(s), where

$$D ( {s}_{i} ) = {\sum }_{j = 1}^{N} dist ( {s}_{i} , {s}_{j} ) .$$

There may be more than one (congruent) centroid sequence C for DNA sequences having identical measures of distance.

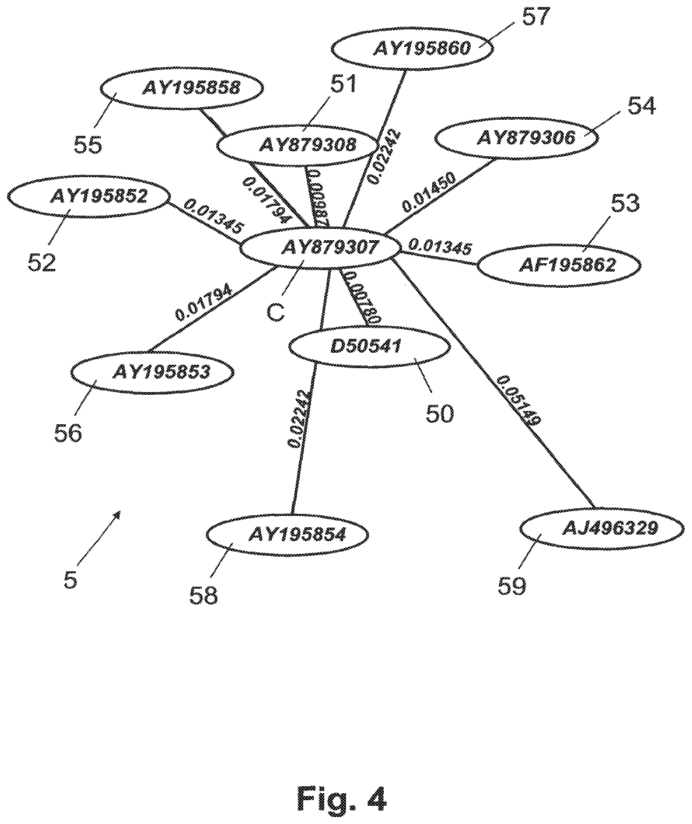

FIG. 4 shows an example of ten DNA sequences 50-59, representing “Abiotrophia defectiva” as shown in FIG. 5, with their respective measures of distance distᵢ(x,y) to the centroid sequence C (“AY879307”).

In step S5, the rating module 124 assigns to the (selected) DNA sequences 111 the measure of distance distᵢ(x,y) between the respective DNA sequence i and the centroid sequence C as a quantitative confidence level for the classification annotation assigned to the respective DNA sequence. The smaller the measure of distance associated with a sequence, the higher the likelihood that this particular sequence is close to the centroid and thus carries its annotation correctly. Thus, a small value of the measure of distance distᵢ(x,y) indicates a high level of confidence; whereas a great value of the measure of distance distᵢ(x,y) indicates a low level of confidence. One skilled in the art will understand, that the level of confidence assigned to the (selected) DNA sequences 111 may alternatively be expressed as a complimentary quantitative value of the measure of distance distᵢ(x,y) or as a qualitative confidence value derived from the measure of distance distᵢ(x,y), e.g. from a set of verbal attributes (e.g. “very high”, “high”, “medium”, “low”, “very low”) or a set of colors.

In optional step S6, the error detector 125 identifies outliers among the DNA sequences of a species. Outliers have the greatest measure of distance to the centroid sequence C of the respective species. For example, in FIG. 4, DNA sequence 59 (“AJ496329”) would be detected as an outlier. In an embodiment, any DNA sequence having a measure of distance to the centroid sequence C above a defined threshold or standard deviation is determined an outlier. In an embodiment, outliers are identified and removed, before determining the centroid sequences (again).

Subsequently, in step S7, the error detector 125 determines whether or not a detected outlier has a smaller measure of distance to a centroid sequence of another species. If that is the case, in step S8, the classification annotation of the outlier is marked as incorrect in reference database 11, e.g. by setting a flag field. In addition, in an embodiment, the classification annotation of the closer centroid sequence is stored assigned to the outlier as a proposed classification annotation.

In a further optional step S9, aside from outliers, the centroid detector 123 assigns the classification annotation associated with a centroid sequence C to the DNA sequences 50-58 associated with that centroid sequence C.

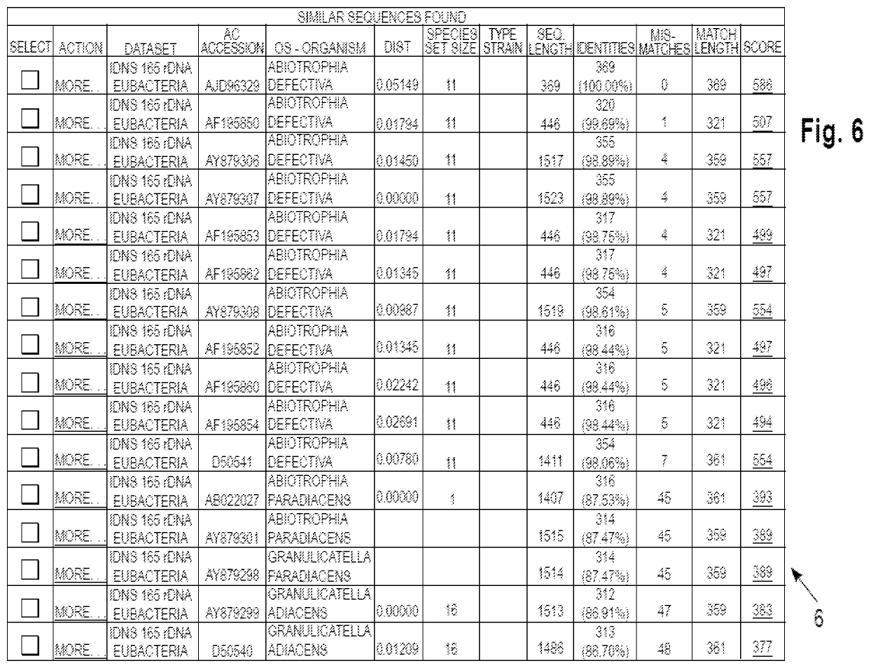

If a user accesses computer system 1 to search the reference database 11 with an uploaded DNA sequence sample, e.g. using sequence data of DNA fragments from a DNA sample from a sequencer 4 or from another source, the user is shown a user interface with a list of possible matches 6 as shown in FIG. 6, for example. As can be seen in FIG. 6, each list entry is provided with its respective measure of distance (dist) to the centroid C as an indicator of the level of confidence. Typically, the list is presented with a ranking by similarity and the level of confidence is used by a user as a measure of reliability of the respective classification annotation. Furthermore, outliers can be visually marked in the list, e.g. through highlighting or coloring, selectively shown or hidden from the list, and alternative classification annotations having a better confidence level can be displayed, e.g. as a proposal of a more suitable classification. The level of confidence values can further be included and displayed in any groupings, alignments, or ranked lists of DNA sequences as well as in phylogenetic trees, for example.

FIG. 3 shows an exemplary sequence of steps for an extended mode of determining the centroid sequences of the (selected) DNA sequences 111. In essence, step S40 is an alternative or complementary approach to the centroid detection performed in step S4. Processing of step S40 may be triggered upon user selection or detection of a level of complexity by the centroid detector 123. The level of complexity may be indicated, for example, by at least a defined number of DNA sequences which have a measure of distance therein between exceeding a complexity threshold.

In step S401, using the scores of similarity stored in the matrix, the graph generator 126 generates an edge-weighted graph 5. The nodes in the graph are representative of the (selected) DNA sequences C, 50-59. Initially, the nodes are connected, if the score of similarity between the respective DNA sequences is positive, i.e. if it is not zero. An initial connectivity threshold may be set for the score of similarity to ensure that the nodes form one coherent graph. A measure of distance between the respective DNA sequences is assigned in each case as an edge weight between the respective nodes. The measure of distance is calculated, for example, as described above in the context of step S41.

In step S402, the graph generator 126 computes the local connectivity densities for the nodes in the graph. The local connectivity density of a node is defined by the number of connections to other nodes in the graph.

In step S403, the graph generator 126 defines clusters of nodes in the graph. The clusters are defined through progressive aggregation to local connectivity density maxima in the graph. Essentially, the measure of distance between DNA sequences associated with nodes within a cluster are significantly shorter than an average measure of distance between the DNA sequences associated with the nodes of the graph. An initial cluster threshold (allowing a large intra-cluster distance) may be defined for the measure of distance between DNA sequences associated with nodes of a cluster so that the whole graph forms just one cluster.

In step S404, the cluster is shown through user interface 1211 to a user on display 33 of data entry terminal 3.

In step S405, optionally, an alternative value for the cluster threshold is received through user interface 1211 from the user at the data entry terminal 3. If it is determined in step S406 that a new cluster threshold was received from the user, the graph generator 126 defines the clusters in step S403 using the new cluster threshold as a maximum intra-cluster distance. Subsequently, the graph with the newly defined cluster is displayed in step S404. If it is determined in step S406 that no new cluster threshold was received from the user, processing continues in step S407.

In step S407, the centroid detector 123 determines the centroid sequence(s) C for the one or more clusters of the graph. For each cluster, the centroid detector 123 determines the DNA sequence associated with the node having the highest connectivity density in the cluster as the centroid sequence C of that cluster. Subsequently processing continues in step S5 as described above with reference to FIG. 2.

It should be noted that, in the description, the computer program code has been associated with specific functional modules and the sequence of the steps has been presented in a specific order, one skilled in the art will understand, however, that the computer program code may be structured differently and that the order of at least some of the steps could be altered, without deviating from the scope of the invention. It should also be noted that the proposed method and system cannot only be used for off-line assessment of classification annotations in a database, but also online (real-time or near real-time), e.g. as a filter for entering the classification annotation for a new DNA sequence to be added to a database.

1. A method of identifying a centroid deoxyribonucleic acid (DNA) sequence of one or more organisms comprising:
obtaining a plurality of DNA sequences from one or more organisms, wherein each DNA sequence is annotated with a classification annotation for one or more taxonomies, systems, and functions related to the DNA sequence;
grouping the plurality of DNA sequences into a plurality of groups based on the classification annotations, wherein each group of the plurality of groups is associated with a different classification annotation;
selecting a group of the plurality of groups;
aligning the DNA sequences of the selected group;
after aligning the DNA sequences in the selected group, determining a measure of distance for each pair of DNA sequences in the selected group based on similarity between the DNA sequences in the pair;
determining, for the selected group, a centroid sequence that has a shortest aggregate measure of distance over all the DNA sequences in the selected group; and
displaying the determined centroid sequence.

2. The method according to claim 1, further comprising:
identifying an outlier DNA sequence within the selected group, wherein the outlier DNA sequence has a greatest measure of distance to the determined centroid sequence;
determining whether the outlier DNA sequence has a measure of distance to a centroid sequence of a group other than the selected group that is smaller than the greatest measure of distance; and
after determining that the outlier DNA sequence has a measure of distance to the centroid sequence of the group other than the selected group smaller than the greatest measure of distance, marking the classification annotation of the outlier DNA sequence as incorrect.

3. The method according to claim 1, further comprising:
generating an edge-weighted graph, wherein the DNA sequences are represented by nodes in the edge-weighted graph, wherein a pair of nodes are connected by an edge of the edge-weighted graph when a score of similarity between the respective DNA sequences is positive, and wherein each edge of the edge-weighted graph has an edge weight based on the measure of distance between the DNA sequences represented by nodes connected by the edge;
computing local connectivity densities for the nodes in the edge-weighted graph; and
defining clusters of nodes through progressive aggregation to local connectivity density maxima.

4. The method according to claim 3, wherein the method further comprises:
displaying the edge-weighted graph using a display associated;
after displaying the edge-weighted graph, receiving a cluster threshold;
defining the clusters of nodes by applying the cluster threshold as a maximum intra-cluster distance; and
after applying the cluster threshold, redisplaying the edge-weighted graph on the display.

5. The method according to claim 3, further comprising:
determining a node of the edge-weighted graph having a highest connectivity density in a selected cluster of the clusters of nodes; and
determining a centroid sequence of the selected cluster to be a DNA sequence associated with the node of the edge-weighted graph having the highest connectivity density in the selected cluster.

6. The method according to claim 1, wherein a classification annotation annotating a centroid sequence of a particular group of the plurality of groups is used to annotate other classification annotations of DNA sequences in the particular group.

7. The method according to claim 1, wherein determining the measure of distance for each pair of DNA sequences in the selected group comprises:
determining a smaller length of the two DNA sequences, and
calculating a weighted score of similarity by at least dividing a score of similarity between the two DNA sequences by the smaller length of the two DNA sequences.

8. The method according to claim 6, wherein the classification annotation annotating the centroid sequence of the particular group comprises a viral group annotation, and wherein the viral group annotation is used to annotate the other classification annotations of DNA sequences in the particular group.

9. The method according to claim 6, wherein the classification annotation annotating the centroid sequence of the particular group comprises a genus name, and wherein the genus name is used to annotate the other classification annotations of DNA sequences in the particular group.

10. The method according to claim 6, wherein the classification annotation annotating the centroid sequence of the particular group comprises a species name, and wherein the species name is used to annotate the other classification annotations of DNA sequences in the particular group.

11. The method according to claim 10, wherein the species name comprises a bacterial species name.

12. The method according to claim 1, wherein determining the measure of distance dist(x,y) between each pair of DNA sequences x and y is calculated by determining a complementary value of a score of similarity score(x,y).

13. The method according to claim 12, wherein the measure of distance is calculated by determining a weighted score of similarity being calculated according to the formula dist(x,y)=1−score(x,y)/min(lₓ,l_{y}) where lₓ and l_{y} are the respective lengths of the pair of DNA sequences.

14. The method according to claim 1, wherein the determining the centroid sequence c of a set of sequences S comprises calculating whether, for all N sequences s in set S different from c, D(c)<D(s), where D(s₁)=Σⱼ₌₁^{N} dist (sᵢ,sⱼ).

15. A computer-readable medium having computer program code stored therein, wherein the computer program code is executable by one or more processors to perform a method of identifying a centroid deoxyribonucleic acid (DNA) sequence of one or more organisms comprising:
obtaining a plurality of DNA sequences from one or more organisms, wherein each DNA sequence is annotated with a classification annotation for one or more taxonomies, systems, and functions related to the DNA sequence;
grouping the plurality of DNA sequences into a plurality of groups based on the classification annotations, wherein each group of the plurality of groups is associated with a different classification annotation;
selecting a group of the plurality of groups;
aligning the DNA sequences of the selected group;
after aligning the DNA sequences in the selected group, determining a measure of distance for each pair of DNA sequences in the selected group based on similarity between the DNA sequences in the pair;
determining, for the selected group, a centroid sequence that has a shortest aggregate measure of distance over all the DNA sequences in the selected group; and
displaying the determined centroid sequence.

16. A computer system configured to identify a centroid deoxyribonucleic acid (DNA) sequence of one or more organisms comprising:
a plurality of DNA sequences obtained from one or more organisms, wherein each DNA sequence is annotated with a classification annotation for one or more taxonomies, systems, and functions related to the DNA sequence;
a comparator module configured to:
group the plurality of DNA sequences into a plurality of groups based on the classification annotations, wherein each group of the plurality of groups is associated with a different classification annotation; and
align the respective DNA sequences of a selected group of the plurality of groups;
a centroid detector configured to:
determine a measure of distances for each pair of DNA sequences in the selected group based on similarity between the DNA sequences in the pair; and
determine a centroid sequence for the selected group, wherein the centroid sequence has a shortest aggregate measure of distance over all the DNA sequences in the selected group; and
a rating module configured to assign a quantitative confidence level for each DNA sequence in the selected group regarding the classification annotation assigned to each DNA sequence and based on the measure of distance between the DNA sequence and the centroid sequence.

17. The computer system according to claim 16, further comprising:
an error detector configured to:
identify an outlier DNA sequence within the selected group having a greatest measure of distance to the centroid sequence;
determine whether the outlier DNA sequence has a measure of distance to a centroid sequence of a group other than the selected group that is smaller than the greatest measure of distance; and
mark the classification annotation of the outlier DNA sequence as incorrect.

18. The computer system according to claim 16, further comprising:
a graph generator configured to:
generate from the similarity an edge-weighted graph, wherein the DNA sequences are represented by nodes in the edge-weighted graph, wherein a pair of the nodes are connected by an edge of the edge-weighted graph when a similarity between the respective DNA sequences is positive, and wherein each edge of the edge-weighted graph has an edge weight based on the measure of distance between DNA sequences represented by nodes connected by the edge;
compute local connectivity densities for the nodes in the edge-weighted graph; and
define clusters of nodes through progressive aggregative to local connectivity density.

19. The computer system according to claim 18, further comprising:
a user interface configured to:
display the edge-weighted graph;
after displaying the edge-weighted graph, receive a cluster threshold;
define the clusters of nodes by applying the cluster threshold as a maximum intra-cluster distance; and
after applying the cluster threshold, redisplay the edge-weighted graph on the display.

20. The computer system according to claim 18, wherein the centroid detector is further configured to:
determine a node of the edge-weighted graph having a highest connectivity density in a designated cluster of the clusters of nodes; and
determine a centroid sequence of the designated cluster to be a DNA sequence associated with the node of the edge-weighted graph having the highest connectivity density in the designated cluster.

21. The computer system according to claim 16, wherein the centroid detector is further configured to annotate DNA sequences in a particular group of the plurality of groups using a classification annotation annotating a centroid sequence of the particular group.

22. The computer system according to claim 16, wherein the comparator module is further configured to determine the measure of distance for each pair of DNA sequences in the selected group by at least:
determining a smaller length of the two DNA sequences, and
calculating a weighted score of similarity by at least dividing a score of similarity between the two DNA sequences by the smaller length of the two DNA sequences.

23. The computer system according to claim 16, further comprising a sequencing device configured to amplify and sequence one or more new DNA sequences of one or more organisms.

24. The computer system according to claim 16, further comprising a data entry terminal configured to enter search requests and display results from the search requests.

25. A method, comprising:
accessing, via a user computer, a centroid database containing one or more centroid sequences determined according to the method of claim 1;
obtaining at least one DNA sequence sample from one or more organisms;
submitting, at the user computer, a search request to search the at least one DNA sequence sample from one or more organisms against the database; and
reviewing one or more entries for one or more DNA sequences of the database that match the DNA sequence sample,
wherein each entry for a DNA sequence of the database comprises: a respective measure of the distance of the DNA sequence to the centroid sequence carrying the same classification annotation assigned to the DNA sequence and a measure of the level of confidence that the classification annotation assigned to the DNA sequence is correct, and
wherein submitting the search request comprises transmitting data from the user computer through a telecommunications network.

26. The method according to claim 25, wherein obtaining the at least one DNA sequence sample comprises obtaining the one or more sample DNA sample sequences using a sequencing device.

27. The method according to claim 25, further comprising adding the one or more sample DNA sequences with the assigned classification annotations to a second database.

28. A computer program memory having stored therein instructions, wherein the instructions comprise code means for carrying out the method according to claim 25.


SEQUENCE LISTING (11 sequences)

SEQ ID NO: 1 - DNA, 180 bp, Abiotrophia defectiva
agtcgaacgaaccgcgactaggtgcttgcacttggtcaaggtgagtggcgaacgggtgagtaacacgtgggtaacctacctcatagtgggggataacagtcggaaacgactgctaataccgcataggacatggaatcacatgattccgtgaggaaaggtggcgcaagctatcgctaagag

SEQ ID NO: 2 - DNA, 180 bp, Abiotrophia defectiva
agtcgaacgaaccgcgactgagtgcttgcacttggtcaaggtgagtggcgaacgggtgagtaacacgtgggtaacctacctcatagtgggggataacagtcggaaacgactgctaataccgcataggacatggaatcacatgattctgtgaggaaaggtggcgcaagctatcgctaagag

SEQ ID NO: 3 - DNA, 180 bp, Abiotrophia defectiva
agtcgaacgaaccgcgactgagtgcttgcacttggtcaaggtgagtggcgaacgggtgagtaacacgtgggtaacctacctcatagtgggggataacagtcggaaacgactgctaataccgcataggacataggatcacatgatccagtgaggaaaggtggcgcaagctatcgctaagag

SEQ ID NO: 4 - DNA, 180 bp, Abiotrophia defectiva
agtcgaacgaaccgcgactgagtgcttgcacttggtcaaggtgagtggcgaacgggtgagtaacacgtgggtaacctacctcatagtgggggataacagtcggaaacgactgctaataccgcataggacatggaatcacatgattccgtgaggaaaggtggcgcaagctatcgctaagag

SEQ ID NO: 5 - DNA, 180 bp, Abiotrophia defectiva
agtcgaacgaaccgcgactaggtgcttgcacttggtcaaggtgagtggcgaacgggtgagtaacacgtgggtaacctacctcatagtgggggataacagtcggaaacgactgctaataccgcataggacatgggatcacatgattctgtgaggaaaggtggcgcaagctatcgctaagag

SEQ ID NO: 6 - DNA, 180 bp, Abiotrophia defectiva
  modified_base (20)..(20): a, c, t, g, unknown or other
  misc_feature (20)..(20): n is a, c, g, or t
agtcgaacgaaccgcgactnagtgcttgcacttggtcaaggtgagtggcgaacgggtgagtaacacgtgggtaacctacctcatagtgggggataacagtcggaaacgactgctaataccgcataggacataggatcacatgatccagtgaggaaaggtggcgcaagctatcgctaagag

SEQ ID NO: 7 - DNA, 180 bp, Abiotrophia defectiva; identical to SEQ ID NO: 2

SEQ ID NO: 8 - DNA, 180 bp, Abiotrophia defectiva
agtcgaacgaaccgcgaccgagtgcttgcacttggtcaaggtgagtggcgaacgggtgagtaacacgtgggtaacctacctcatagtgggggataacagtcggaaacgactgctaataccgcataggacatgggatcacatgattccgtgaggaaaggtggcgcaagctatcgctaagag

SEQ ID NO: 9 - DNA, 180 bp, Abiotrophia defectiva
agtcgaacgaaccgcgactgagtgcttgcacttggtcaaggtgagtggcgaacgggtgagtaacacgtgggtaacctacctcatagtgggggataacagtcggaaacgactgctaataccgcataggacatggratcacatgattccgtgaggaaaggtggcgcaagctatcgctaagag

SEQ ID NO: 10 - DNA, 180 bp, Abiotrophia defectiva
  modified_base (20)..(20): a, c, t, g, unknown or other
  misc_feature (20)..(20): n is a, c, g, or t
  modified_base (34)..(34): a, c, t, g, unknown or other
  misc_feature (34)..(34): n is a, c, g, or t
agtcgaacgaaccgcgactnagtgcttgcactangtcaaggtgagtggcgaacgggtgagtaacacgtgggtaacctacctcatagtgggggataacagtcggaaacgactgctaataccgcataggacatggaatcacatgattccgtgaggaaaggtggcgcaagctatcgctaagag

SEQ ID NO: 11 - DNA, 180 bp, Abiotrophia defectiva
  modified_base (134)..(134): a, c, t, g, unknown or other
  misc_feature (134)..(134): n is a, c, g, or t
agtcgaacgaaccgcgactaggtgcttgcacttggtcaaggtgagtggcgaacgggtgagtaacacgtgggtaacctacctcatagtgggggataacagtcggaaacgactgctaataccgcataggacatggnatcacatgattcagtgaggaaaggtggcgcaagctatcgctaagag